(19) United States
(12) Patent Application Publication
(10) Pub. No.: US 2025/0114551 A1
(43) Pub. Date: Apr. 10, 2025

(54) CANNULA FOR ORAL AND NASAL OXYGEN SUPPLY AND CAPNOGRAPHY

(71) Applicant: Oridion Medical 1987 Ltd., Jerusalem (IL)

(72) Inventors: Denis Glozman, Modiin (IL); Alexander Rabkin, Mevaseret Zion (IL); Shai Fleischer, Modiin (IL); Yedidia Blonder, Jerusalem (IL)

(21) Appl. No.: 18/828,704

(22) Filed: Sep. 9, 2024

Related U.S. Application Data
(60) Provisional application No. 63/664,994, filed on Jun. 27, 2024.
(60) Provisional application No. 63/542,446, filed on Oct. 4, 2023.

(51) Int. Cl. A61M 16/06; A61M 16/08

(52) U.S. Cl. CPC A61M 16/0677; A61M 16/0841; A61M 2210/0618

(57) ABSTRACT

A cannula for exhaled gas monitoring and oxygen delivery includes an oxygen delivery conduit entering the cannula and an exhalation conduit exiting the cannula. The cannula further includes first and second nasal prongs each comprising a channel directing exhaled nasal breath into the exhalation conduit, an oral scoop forming a cavity that is fluidly coupled to the exhalation conduit, and first and second ports fluidly coupled to the oxygen delivery conduit and opening toward the oral scoop.

### CROSS-REFERENCE TO RELATED APPLICATIONS

This application claims priority to and the benefit of U.S. Provisional Application No. 63,664,994, filed Jun. 27, 2024, entitled “CANNULA FOR ORAL AND NASAL OXYGEN SUPPLY AND CAPNOGRAPHY,” and U.S. Provisional Application No. 63/542,446, filed Oct. 4, 2023, entitled “CANNULA FOR ORAL AND NASAL OXYGEN SUPPLY AND CAPNOGRAPHY,” each of which is hereby incorporated by reference in its entirety for all purposes.

### TECHNICAL FILED

The present disclosure generally relates to a capnography cannula that delivers oxygen orally and nasally and that permits sampling of exhaled carbon dioxide (CO₂).

### BACKGROUND

Different types of nasal cannulas are used to deliver oxygen to patients who require assistance to breathe properly, to collect carbon dioxide samples from patients to monitor respiration, or to perform both functions. Such cannulas may be used when direct ventilation is not provided. A nasal cannula provides oxygen supply to the nasal cavity. Some nasal cannulas have breath sampling capability such that a sample of the patient's exhaled air flows through the cannula to a gas analyzer to be analyzed. The results of this non-invasive analysis provide an indication of the patient's condition, such as the state of the patient's pulmonary perfusion, respiratory system, and metabolism.

An example of a gas analysis often performed is capnography using an analyzer called a capnograph. Capnography is the monitoring of the time-dependent respiratory carbon dioxide (CO₂) concentration, which may be used to directly monitor the inhaled and exhaled concentration of CO₂ and indirectly monitor the CO₂ concentration in a patient's blood. Capnography may provide information about CO₂ production, pulmonary (lung) perfusion, alveolar ventilation (alveoli are hollow cavities in the lungs in which gas exchange is being performed) and respiratory patterns. Capnography may also provide information related to a patient's condition during anesthesia, for example by monitoring the elimination of CO₂ from anesthesia breathing circuit and ventilator.

### SUMMARY

Certain embodiments commensurate in scope with the originally claimed subject matter are summarized below. These embodiments are not intended to limit the scope of the disclosure. Indeed, the present disclosure may encompass a variety of forms that may be similar to or different from the embodiments set forth below.

In one embodiment, a cannula for exhaled gas monitoring and oxygen delivery includes an oxygen delivery conduit entering the cannula and an exhalation conduit exiting the cannula. The cannula further includes first and second nasal prongs each comprising a channel directing exhaled nasal breath into the exhalation conduit, an oral scoop forming a cavity that is fluidly coupled to the exhalation conduit, and first and second ports fluidly coupled to the oxygen delivery conduit and opening toward the oral scoop.

In another embodiment, a capnography system includes a cannula that includes an oxygen delivery conduit entering the cannula, an exhalation conduit exiting the cannula, an oral scoop forming a cavity that is fluidly coupled to the exhalation conduit, and first and second oral oxygen delivery ports fluidly coupled to the oxygen delivery conduit and opening toward the oral scoop. The capnography system also includes a monitor fluidly coupled to the exhalation conduit to analyze exhaled carbon dioxide received via the exhalation conduit.

In another embodiment a method of capnography includes receiving, via an oral scoop of a cannula, an exhaled breath from a patient, where the exhaled breath includes carbon dioxide, and where the oral scoop is positioned proximate an oral cavity of the patient in an installed configuration of the cannula device. The method further includes directing, via a sampling line coupled to the cannula device, the exhaled breath to a gas analyzer, and delivering, via one or more oral oxygen delivery ports of the cannula device, oxygen from an oxygen source to the oral cavity of the patient while receiving the exhaled breath.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and context of embodiments of the present disclosure without limitation to the claimed subject matter.

### BRIEF DESCRIPTION OF THE DRAWINGS

Advantages of the disclosed techniques may become apparent upon reading the following detailed description and upon reference to the drawings in which:

FIG. 1 is a schematic view of an embodiment of a capnography system, in accordance with an aspect of the present disclosure;

FIG. 2 is a perspective view of an embodiment of a cannula which may be employed in the capnography system of FIG. 1, in accordance with an aspect of the present disclosure;

FIG. 3A is a perspective view of an embodiment of a cannula which may be employed in the capnography system of FIG. 1, in accordance with an aspect of the present disclosure;

FIG. 3B is a side view of an embodiment of the cannula of FIG. 3A, in accordance with an aspect of the present disclosure;

FIG. 4A is a perspective view of an embodiment of the cannula of FIG. 3A, illustrating oxygen delivery flow paths, in accordance with an aspect of the present disclosure;

FIG. 4B is a perspective view of an embodiment of the cannula of FIG. 3A, illustrating exhaled CO₂ flow paths, in accordance with an aspect of the present disclosure;

FIG. 5A is a perspective view of an embodiment of a cannula which may be employed in the capnography system of FIG. 1, in accordance with an aspect of the present disclosure;

FIG. 5B is a side view of an embodiment of the cannula of FIG. 6A, in accordance with an aspect of the present disclosure;

FIG. 6A is a perspective view of an embodiment of a cannula which may be employed in the capnography system of FIG. 1, in accordance with an aspect of the present disclosure;

FIG. 6B is a side view of an embodiment of the cannula of FIG. 6A, in accordance with an aspect of the present disclosure;

FIG. 7A is a perspective view of an embodiment of a cannula which may be employed in the capnography system of FIG. 1, in accordance with an aspect of the present disclosure;

FIG. 7B is a perspective view of an embodiment of the cannula of FIG. 7A, in accordance with an aspect of the present disclosure;

FIG. 7C is a side view of an embodiment of the cannula of FIG. 7A, in accordance with an aspect of the present disclosure;

FIG. 8A is a perspective view of an embodiment of a cannula which may be employed in the capnography system of FIG. 1, in accordance with an aspect of the present disclosure;

FIG. 8B is a perspective view of an embodiment of the cannula of FIG. 7A, in accordance with an aspect of the present disclosure;

FIG. 8C is a side view of an embodiment of the cannula of FIG. 7A, in accordance with an aspect of the present disclosure;

FIG. 9A is perspective view of an embodiment of a cannula, which may be employed in the capnography system of FIG. 1, in accordance with an aspect of the present disclosure; and

FIG. 9B is a perspective view of an embodiment of the cannula of FIG. 9A, in accordance with an aspect of the present disclosure.

### DETAILED DESCRIPTION OF THE INVENTION

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features.

The present disclosure generally relates to the field of breath monitoring, and more particularly, to CO₂ sampling alongside oral/nasal oxygen delivery using the oral/nasal cannula devices disclosed herein. A nasal cannula may provide a nasal breath sample for capnography via collected exhaled gases that are collected through prongs inserted into nasal cavities. The use of separate prongs for nasal exhalation sampling separates the collection of exhaled nasal gases from the actively delivered oxygen, such that the delivered oxygen does not significantly mix with the sampled nasal gases to permit more accurate measurements. In some cases, patients can only or primarily breathe through the mouth and may exhale through both the nasal cavity and the oral cavity. Thus, sampling exhaled air through the mouth may provide a representation of respiratory function for capnography. However, it can be challenging to both actively supply oxygen to the mouth through a cannula and to accurately measure CO₂ exhaled from the mouth while oxygen is being supplied. For example, when oxygen is continuously directed towards the mouth during oxygen delivery, any delivered oxygen may mix with the exhaled breath from the mouth, thus diluting the gas composition and preventing accurate measurements. While an oral prong to capture exhaled mouth gases may be inserted into the mouth, similarly to a nasal prong, this may be uncomfortable for the patient and may interfere with the advantages of the nasal cannula, which typically permits patients to speak and eat and drink normally.

Certain nasal cannulas may include oxygen delivery openings that are oriented towards the mouth to provide oxygen delivery. However, typically, such oxygen delivery arrangements are done with relatively low flow (e.g., 2-5 L/minute oxygen delivery) and are not used in situations in which higher levels of oxygen delivery are needed and/or when nasal breathing is obstructed. However, if the inspiratory flow rate of the patient is greater than what is being provided by the cannula, the patient will entrain room air into the lungs. This results in oxygen dilution, and the patient will not be receiving the precise amount of oxygen that is desired. While an oxygen mask can provide higher levels of oxygen delivery, capnography measurements are challenging in conjunction with a mask. Accordingly, it is now recognized that improved systems and methods that enable capnometric measurement of exhaled CO₂ while simultaneously providing adequate oxygenation and that may be used with higher flow oxygen delivery (e.g., up to 15 L/minute) to a patient are desired.

Present embodiments are directed to oral/nasal cannulas that are capable of providing oxygen to both a nasal cavity of a patient and to an oral cavity of a patient, thereby providing adequate oxygenation to patients, while also being capable of sampling exhaled carbon dioxide from a nasal cavity and/or an oral cavity of the patient. The cannulas disclosed herein may include an intake inlet configured to couple to an oxygen source. The intake inlet may be fluidly coupled to an oxygen delivery conduit (e.g., first conduit system) configured to direct oxygen from the oxygen source to one or more oral oxygen delivery ports and to one or more nasal oxygen delivery ports. The one or more oral oxygen delivery ports and the one or more nasal oxygen delivery ports may be positioned on opposite sides of the cannula to provide adequate oxygenation to a patient and, in embodiments, may provide capnography. The cannula may also include an oral scoop configured to be positioned proximate an oral cavity of the patient and one or more nasal prongs configured to be inserted into a nasal cavity of the patient. The oral scoop and the one or more nasal prongs may be configured to receive exhaled breath from the patient. The exhaled breath may comprise exhaled carbon dioxide that is to be analyzed by a gas analyzer. As a patient exhales, the oral scoop and/or the one or more nasal prongs may direct the exhaled carbon dioxide into an exhalation conduit (e.g., second conduit system) which may be fluidly coupled to a sampling outlet of the cannula. The sampling outlet of the cannula may be coupled to a sampling line configured to direct the exhaled carbon dioxide to a gas analyzer coupled to an end of the sampling line opposite the end that is coupled to the sampling outlet of the cannula. The oxygen delivery conduit and the exhalation conduit may be fluidly isolated from one another to ensure that oxygen delivered to a patient does not mix with exhaled carbon dioxide received from the patient.

In certain embodiments, the one or more oral oxygen delivery ports may be oriented such that oxygen is delivered into the oral scoop as the patient inhales. During an exhalation phase, the force or pressure from the patient's exhaled breath may redirect the oxygen away from the oral scoop, thereby preventing mixing of the oxygen delivered to the patient and the carbon dioxide received from the patient via the oral scoop and the exhalation conduit. In other embodiments, the one or more oral oxygen delivery ports may be oriented such that oxygen is delivered outside of the oral scoop, thereby limiting an amount of mixing between the oxygen delivered to the patient and the carbon dioxide received from the patient during an exhalation phase. In still other embodiments, the cannula may include one or more plates configured to direct oxygen at an angle toward the oral scoop, as described in greater detail below.

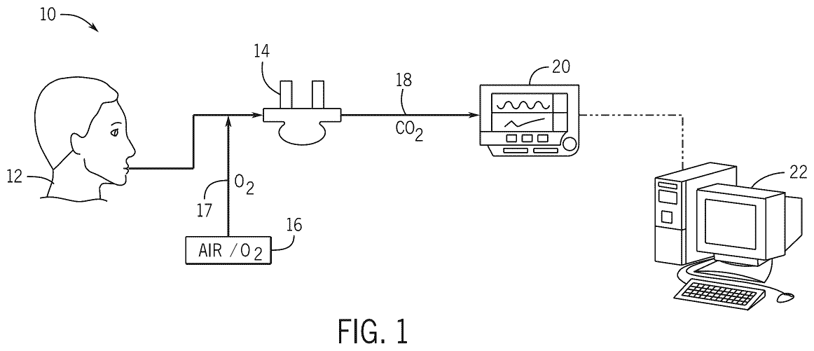

Turning now to FIG. 1, a capnography system 10 is illustrated that may be used in conjunction with the disclosed oral/nasal cannulas provided herein. The capnography system 10 may be utilized to sample exhaled breath from a patient 12, while providing adequate oxygenation to the patient 12. For example, the capnography system 10 may include a cannula 14 configured to provide oxygen to the patient's nasal cavity and oral cavity during inhalation, while simultaneously capturing exhaled CO₂ from the patient 12 during exhalation. In certain embodiments, oxygen provided to the patient 12 via the cannula 14 may be received from an oxygen source 16. The oxygen source 16 may be coupled to an oxygen line 17, and the oxygen line 17 may be coupled to the cannula 14, thereby enabling delivery of oxygen into the cannula 14. The cannula 14 may include a first conduit system configured to receive the oxygen from the oxygen source 16 and deliver the oxygen to the patient 12 via one or more oral oxygen delivery ports and/or one or more nasal oxygen delivery ports, as described in greater detail below.

In certain embodiments, the cannula 14 may be used in conjunction with low flow oxygen delivery or high flow oxygen delivery. In an embodiment, a low flow oxygen delivery may be between 2-6 liters of oxygen per minute (L/min), while a high flow oxygen delivery may deliver oxygen at a rate above 6 L/min or, in embodiments, above 10 L/min. In an embodiment, a high flow oxygen delivery may deliver oxygen at a rate from 10 L/min-15 L/min. The oxygen delivery may be continuous flow or intermittent flow.

As the patient exhales, CO₂ may be captured by the cannula 14 and delivered to a gas analyzer 20 for analysis via a sampling line 18. For example, the cannula 14 may include one or more nasal prongs configured to be inserted into a nasal cavity of the patient 12 and an oral scoop configured to be positioned proximate an oral cavity of the patient 12. The one or more nasal prongs and the oral scoop may be fluidly coupled to a second conduit system of the cannula 14, thereby enabling capture and delivery of exhaled CO₂ to the gas analyzer 20 via the sampling line 18. The gas analyzer 20 (e.g., capnograph) may provide an indication of the patient's health based on measurement of the CO₂ in sampled gas received from the cannula 14. In certain embodiments, the gas analyzer 20 may be communicatively coupled to a computer 22 (e.g., a desktop computer, a tablet, a mobile device) or a separate display device to facilitate the display of information related to the patient's condition, which may include displayed CO₂ measurements from the gas analyzer.

The system 10 may include additional or fewer components than those illustrated. In certain embodiments, the gas analyzer 20 and the computer 22 may be integrated into a single unit. Further, the illustrated oxygen line 17 and/or the sampling line 18 may include one or more gas transfer conduits. For example, the oxygen line 17 may include one or more fluidically connected conduits, and/or the sampling line 18 may include one or more fluidically connected conduits.

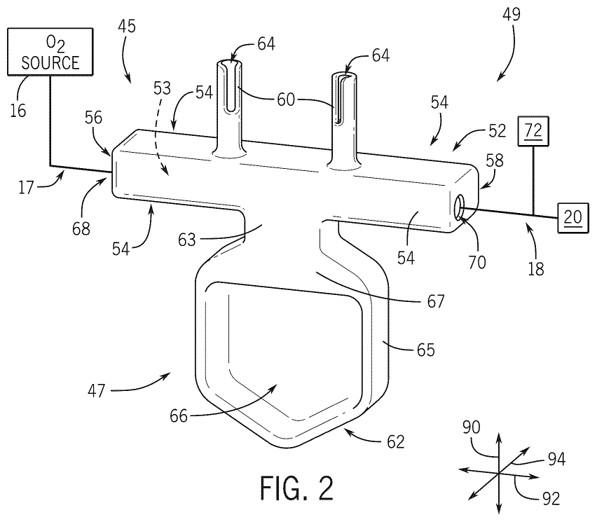

FIG. 2 is a perspective view of an embodiment of a cannula 45 (e.g., cannula 14) which may be employed by the capnography system 10 of FIG. 1. The cannula 45 permits oral oxygen and nasal oxygen delivery (e.g., continuous oxygen delivery) and sampling of exhaled nasal gases and exhaled mouth gases via the cannula 45. As illustrated, the cannula 45 (e.g., cannula device, oral/nasal cannula) has a patient-facing side 47 oriented toward the patient in an assembled configuration of the cannula 45, and an outer side 49 (e.g., environment-facing side) oriented away from the patient (e.g., oriented toward the environment) in the assembled configuration of the cannula 45. The cannula 45 may include a housing 52 defining an interior space 53 of the cannula 45. The housing 52 may be defined by a plurality of sides 54, and may include a first end 56 and a second end 58. The cannula 45 includes a pair of nasal prongs 60 extending from a first side 54 (e.g., top side, upper side) of the housing 52 in a direction (e.g., upward direction) along a vertical axis 90 of the cannula 45. In certain embodiments, each of the nasal prongs 60 may extend at an angle in a direction (e.g., upward direction) relative to the vertical axis 90 toward the nasal cavity of the patient (e.g., toward the patient-facing side 47) in an assembled configuration of the cannula 45. The cannula 45 also includes an oral scoop 62 extending from a second side 54 (e.g., bottom side) of the housing 52 in a direction (e.g., downward direction) along the vertical axis 90 of the cannula 45. In certain embodiments, the oral scoop 62 may extend at an angle in a direction (e.g., downward direction) relative to the vertical axis 90 toward an oral cavity (e.g., mouth) of the patient (e.g., toward the patient-facing side 47) in an assembled configuration of the cannula 45. Each nasal prong 60 of the pair of nasal prongs 60 may be configured to be inserted into a nasal cavity of the patient when the cannula is in use with a patient (see FIG. 1), and each nasal prong 60 may define a channel 64 configured to receive exhaled breath from the nasal cavity of the patient when in use. The exhaled breath received by the nasal prongs 60 may include CO₂, which may be measured as part of capnography monitoring as provided herein.

In certain embodiments, the oral scoop 62 may be configured to be positioned proximate an oral cavity (e.g., mouth) of the patient, thereby permitting the oral scoop 62 to capture exhaled breath from the oral cavity of the patient. For example, the oral scoop 62 may include a neck 63 (e.g., neck portion) extending from the housing 52 and a body portion 65 (e.g., body) which may define a cavity 66 configured to receive exhaled breath from the oral cavity of the patient, and the exhaled breath may include carbon dioxide. In certain embodiments, the body 65 may include a lip portion 67 that further defines the cavity 66. For example, the lip portion 67 may at least partially extend from the neck 63 into the cavity 66. In certain embodiments, the lip portion 67 may be configured to facilitate delivery of oxygen into the oral cavity of a patient and/or limit an amount of mixing between the oxygen delivered to the oral cavity of the patient and the exhaled oral breath received from the patient, as discussed in greater detail below. It should be noted that while the body 65 of the oral scoop 62 is illustrated as having a pentagonal shape, the body 65 of the oral scoop 62 may take on other shapes including square, triangular, circular, ovoidal or any other suitable shape without departing from the scope of this disclosure. In an embodiment, the scoop 62 is configured to at least in part contact the patient when the cannula 45 is in use.

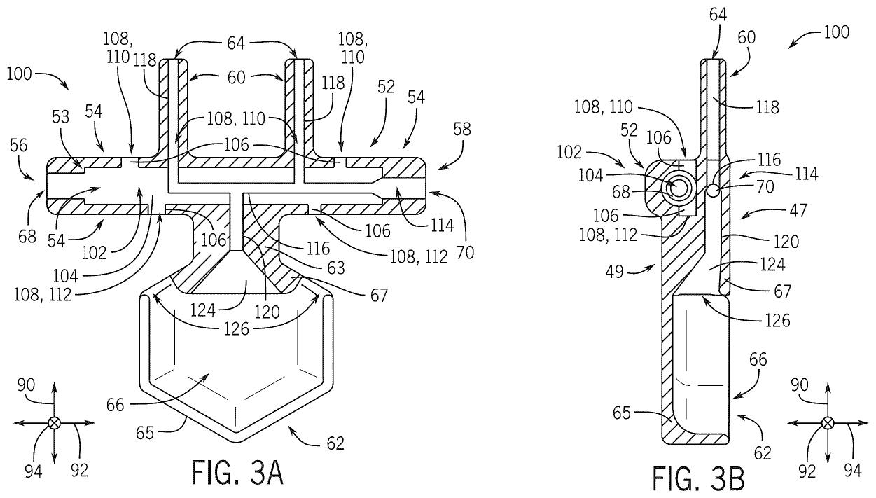

In certain embodiments, the first end 56 of the housing 52 may include an intake inlet 68 fluidly coupled to the oxygen source 16 via the oxygen line 17. The second end 58 of the housing 52 may include a sampling outlet 70 fluidly coupled to the gas analyzer 20 via the sampling line 18. The intake inlet 68 and the sampling outlet 70 may be fluidly coupled to respective conduit systems (see FIG. 3A and FIG. 3B, for example) disposed within the interior space 53 defined by the housing 52. For example, the intake inlet 68 may be fluidly coupled to an oxygen delivery conduit (e.g., first conduit system) entering the cannula 45 and configured to direct oxygen from the oxygen source 16 toward one or more oral oxygen delivery ports and to one or more nasal oxygen delivery ports, as described in greater detail below. The intake inlet 68 and the sampling outlet 70 may include suitable couplings or connectors to permit reversible attachment of conduits (e.g., tubing) to permit transfer of gases from the oxygen supply 16 and to the gas analyzer 20.

The one or more oral oxygen delivery ports may be configured to direct the oxygen in a direction (e.g., downward direction) along the vertical axis 90 of the cannula 45 toward the oral scoop 62 and toward the oral cavity of the patient. The one or more nasal oxygen delivery ports may be configured to direct the oxygen in a direction (e.g., upward direction) along the vertical axis 90 toward the nasal cavity of the patient.

The sampling outlet 70 may be fluidly coupled to an exhalation conduit (e.g., second conduit system) within the interior space 53 that is configured to receive exhaled breath from the nasal cavity of the patient via the nasal prongs 60 and/or from the oral cavity of the patient via the oral scoop 62. For example, the channels 64 defined by the nasal prongs 60 may be fluidly coupled to the exhalation conduit such that as a patient exhales through the nasal cavity, the channels 64 may direct the exhaled nasal breath into the exhalation conduit. In turn, the exhalation conduit may deliver the exhaled nasal breath toward the sampling outlet 70, which may then direct the exhaled nasal breath to the gas analyzer 20 via the sampling line 18. Similarly, the cavity 66 defined by the oral scoop 62 may also be fluidly coupled to the exhalation conduit, thereby enabling exhaled oral breath from the patient to be directed from the oral scoop 62, into the exhalation conduit, and through the sampling outlet 70 toward the gas analyzer 20 via the sampling line 18. Each of the oxygen delivery conduit and the exhalation conduit may be fluidly isolated from one another, thereby limiting an amount of mixing between the oxygen delivered to the patient and the exhaled carbon dioxide received from the patient. In this way, the accuracy of the analysis performed by the gas analyzer 20 may be improved. Additionally, in certain embodiments, the sampling line 18 may be coupled to a suction source 72 (e.g., pump) configured to entrain exhaled breath into the sampling line 18. For example, the suction source 72 may be a pump configured to draw exhaled breath through the exhalation conduit, out of the sampling outlet 70, and toward the gas analyzer 20.

In an embodiment, the cannula 45 is a unitary structure. For example, the cannula 45 may be a molded or formed piece. In other embodiment, the cannula 45 may be assembled from component parts. The cannula housing 52 may be silicon, rubber, plastic, other polymeric material, metal, or any other suitable material(s).

FIGS. 3A and 3B illustrate an embodiment of a cannula 100 (e.g., cannula 14, cannula 45) configured to deliver oxygen to a patient's oral and nasal cavities and receive exhaled carbon dioxide from the patient's oral and nasal cavities. The cannula 100 may include similar features to the cannula 45. For example, the cannula 100 may include the plurality of sides 54 that define the housing 52 and the interior space 53, the first end 56, the second end 58, the nasal prongs 60, the oral scoop 62, the channels 64, the body 65, the cavity 66, the lip portion 67, the intake inlet 68 disposed at the first end 56 of the housing 52, and the sampling outlet 70 disposed at the second end 58 of the housing 52.

As illustrated in FIG. 3A, the cannula 100 may include an oxygen delivery conduit 102 (e.g., first conduit system, oxygen delivery conduit system) fluidly coupled to the intake inlet 68 and configured to receive oxygen from the oxygen source 16. The oxygen delivery conduit 102 may include one or more different conduits configured to direct oxygen to an oral and/or nasal cavity of a patient. For example, the oxygen delivery conduit 102 may include a primary conduit 104 (e.g., main conduit, body conduit) and a plurality of secondary conduits 106 that each terminate at an oxygen delivery port 108. That is, a first set of the secondary conduits 106 may extend in a direction (e.g., upward direction) along the vertical axis 90 toward a nasal cavity of the patient, while a second set of the secondary conduits 106 may extend in a direction (e.g., downward direction) along the vertical axis toward an oral cavity of the patient. Thus, the oxygen delivery ports 108 associated with the first set of secondary conduits 106 may correspond to nasal oxygen delivery ports 110 opening away from the oral scoop 62, and the oxygen delivery ports 108 associated with the second set of secondary conduits 106 may correspond to oral oxygen delivery ports 112 opening toward the oral scoop 62. It should be appreciated that while the secondary conduits 106 and oxygen delivery ports are illustrated as extending directly along the vertical axis 90, in certain embodiments, each of the secondary conduits 106 and the oxygen delivery ports 108 may extend at a respective angle (e.g., non-zero angle) relative to the vertical axis 90 (e.g., in a direction along a longitudinal axis 94 of the cannula 100 toward the oral cavity and nasal cavity of the patient).

Each of the oxygen delivery ports 108 may be sized and configured to enable appropriate distribution of oxygen to the patient. In an embodiment, the oxygen delivery ports 108 may be sized and configured such that fifty percent of the oxygen received from the oxygen source 16 is delivered to the nasal cavity of the patient, while the remaining fifty percent of the oxygen received from the oxygen source 16 is delivered to the oral cavity of the patient, thereby providing adequate oxygenation to the patient. However, other distributions are also contemplated. Additionally, each of the nasal oxygen delivery ports 110 may be sized and configured relative to one another to enable appropriate distribution (e.g., symmetrical distribution) through each of the nasal oxygen delivery ports 110 and each of the oral oxygen delivery ports 112 may be sized and configured relative to one another to enable appropriate distribution (e.g., symmetrical distribution) through each of the oral oxygen delivery ports 112, as discussed in greater detail below. Further, while two nasal oxygen delivery ports 110 are illustrated, it should be understood that more or fewer ports 110 may be used. In an embodiment, one, two, three, four, five or more ports 110 may be present on the cannula 100. Similarly, while two oral oxygen delivery ports 112 are illustrated, it should be understood that more or fewer ports 112 may be used. In an embodiment, one, two, three, four, five or more ports 112 may be present on the cannula 100.

The cannula 100 may also include an exhalation conduit 114 (e.g., second conduit system, exhalation conduit system) exiting the cannula 100 and configured to receive exhaled breath from the nasal cavity and the oral cavity of the patient. Similar to the oxygen delivery conduit 102, the exhalation conduit 114 may include one or more different conduits extending in different directions to facilitate collection of exhaled breath from the patient's oral and/or nasal cavities. For example, the exhalation conduit 114 may include a primary conduit 116 extending in a direction (e.g., horizontal direction) along a lateral axis 92 of the cannula 100, a pair of secondary conduits 118 extending in a direction (e.g., upward direction) along the vertical axis 90 of the cannula 45, and an additional secondary conduit 120 extending in a direction (e.g., downward direction) along the vertical axis 90 of the cannula 100. The first set of secondary conduits 118 may be configured to extend through the channels 64 of the nasal prongs 60, and thus, may be configured to receive nasal exhaled breath from the patient and deliver the nasal exhaled breath to the primary conduit 116 of the exhalation conduit 114. The additional secondary conduit 120 may be configured to extend through the neck 63 of the oral scoop 62, thereby enabling the additional secondary conduit 120 to receive oral exhaled breath from the patient (e.g., oral exhaled breath within the cavity 66) and deliver the oral exhaled breath to the primary conduit 116 of the exhalation conduit 114. In certain embodiments, the additional secondary conduit 120 may be fluidly coupled to a funnel 124 disposed within the oral scoop 62 (e.g., fluidly coupled to the cavity 66). The funnel 124 may be at least partially defined by the body 65 (e.g., by the lip portion 67) of the oral scoop 62 and may be configured to facilitate collection of the oral exhaled breath from the patient for collection and delivery of exhaled breath to the gas analyzer 20 for analysis. It should be appreciated that while the secondary conduits 118 and the additional secondary conduit 120 are illustrated as extending directly along the vertical axis 90, in certain embodiments, each of the secondary conduits 118 and the additional secondary conduit 120 may extend at a respective angle relative to the vertical axis 90 (e.g., in a direction along the longitudinal axis 94).

In certain embodiments, the cannula 100 illustrated in FIGS. 3A and 3B may be configured to deliver oxygen into the oral scoop 62 during an inhalation phase of the patient. For example, each of the oral oxygen delivery ports 112 may be oriented to align with a portion of the oral scoop 62 such that oxygen directed out of the oral oxygen delivery ports 112 is directed directly into the oral scoop 62. To this end, the oral scoop 62 may include one or more openings 126 disposed on an upper side of the oral scoop 62. The openings 126 may substantially align with the oral oxygen delivery ports 112 along the vertical axis 90, thereby enabling delivery of oxygen from the oral oxygen delivery ports 112 and into the cavity 66 of the oral scoop 62. Further, each of the nasal oxygen delivery ports 110 may be positioned proximate the nasal prongs 60. For example, FIGS. 3A and 3B illustrate the nasal oxygen delivery ports 110 being positioned behind the nasal prongs 60 relative to a patient-facing side 128 of the cannula 100. However, in other embodiments, the nasal oxygen delivery ports 110 may be positioned in front of the nasal prongs 60 relative to the patient-facing side 47 of the cannula 100, or adjacent to the nasal prongs 60, thereby enabling oxygen to be delivered into the nasal cavity of the patient. In certain embodiments, the nasal oxygen delivery ports 110 may be positioned a threshold distance away from the nasal prongs 60 of the cannula 100, thereby reducing pressure within the nasal cavity of the patient during inhalation and exhalation. In this way, an amount of mixing between oxygen delivered via the nasal oxygen delivery ports 110 and nasal exhaled breath may be reduced and a patient experience may be improved. It should be appreciated that a cannula 100 having a particular nasal oxygen delivery port 110 configuration (e.g., arrangement of nasal oxygen delivery ports relative to the nasal prongs 60) may be selected for a patient based on certain physical characteristics of the patient (e.g., size of face, facial structure, facial geometry).

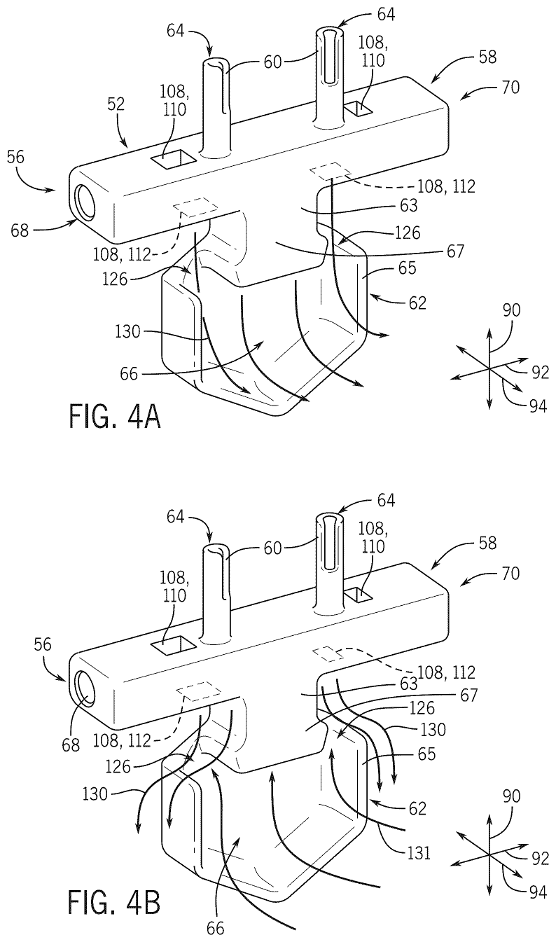

FIGS. 4A and 4B illustrate the movement of delivered gas and exhaled gas through the oral scoop 62 of the cannula 100 during inhalation and exhalation, respectively. In particular, FIGS. 4A and 4B illustrate the movement of delivered gas into the scoop 62 as in the example shown in FIGS. 3A and 3B. For example, as illustrated in FIG. 4A, during an inhalation phase, the flow of oxygen, shown by arrows 130, may be delivered toward the oral scoop 62. The oxygen may be delivered as part of a continuous oxygen delivery. As noted above, the oral scoop 62 of the cannula 100 may include openings 126 configured to align with the oral oxygen delivery ports 112 along the vertical axis 90 such that oxygen traveling out of the oral oxygen delivery ports 112 is directed through the openings 126 and into the cavity 66 of the oral scoop 62. In this way, the oxygen may be delivered to the oral cavity of the patient. In certain embodiments, the oral oxygen delivery ports 112 may be configured to deliver oxygen at a desired flow rate into the cavity 66 of the oral scoop 62, as described in greater detail below.

The desired flow rate of oxygen through the oral oxygen delivery ports 112 may be based on an expected pressure or flow of an exhaled breath from an oral cavity of the patient. For example, FIG. 4B illustrates the redirection of the flow of oxygen 130 relative to the scoop 66 as a patient exhales. The flow of oxygen 130 from the oral oxygen delivery ports 112 may be overcome by a pressure of the exhaled breath 131 from the patient. Thus, as the patient exhales, oxygen may be pushed or redirected out of the oral scoop 62 by the patient's exhaled breath 131. That is, the pressure of exhaled breath 131 from the patient's oral cavity may be greater than the pressure oxygen delivered to the oral cavity of the patient via the oral oxygen delivery ports 112. In this way, any oxygen which may be present within the oral scoop 62 during an exhalation phase of a patient may be redirected or biased out of the oral scoop 62, thereby limiting dilution of the exhaled breath 131 received by the exhalation conduit 114 and delivered to the gas analyzer 20. Further, the sampled exhaled breath 131 does not mix with incoming delivered oxygen during exhalation. In this manner, oxygen influx into the scoop 62 is temporarily overcome by the exhaled breath 131 pushing the oxygen out and around the scoop 62. This serves to separate the sampled exhaled breath 131 from the incoming flow of oxygen 130, permitting sampling of exhaled breath 131 from the mouth while simultaneously delivering oxygen towards the mouth.

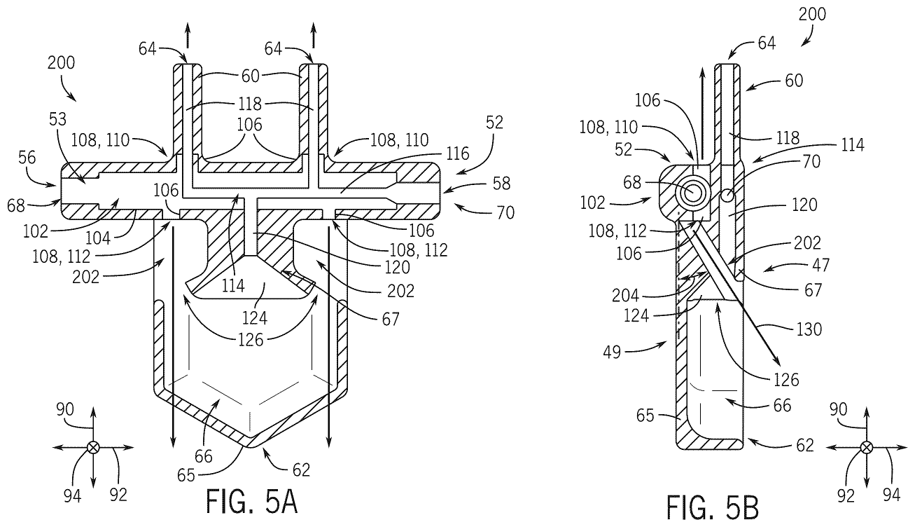

FIGS. 5A and 5B illustrate an embodiment of a cannula 200 (e.g., cannula 14, cannula 45) configured to deliver oxygen to a patient's oral and nasal cavities. The cannula 200 may include similar features to the cannula 45. For example, the cannula 200 may include the plurality of sides 54 that define the housing 52 and the interior space 53, the first end 56, the second end 58, the nasal prongs 60, the oral scoop 62, the channels 64, the body 65, the cavity 66, the lip portion 67, the intake inlet 68 disposed at the first end 56 of the housing 52, and the sampling outlet 70 disposed at the second end 58 of the housing 52. Additionally, the cannula 200 may include similar features to the cannula 100. For example, the cannula 200 may include the oxygen delivery conduit 102 (e.g., second conduit system, oxygen delivery conduit system) configured to receive oxygen from the oxygen source 16 and deliver the oxygen to the patient's oral and nasal cavities. Thus, the cannula 200 may include the primary conduit 104, the first set of secondary conduits 106 extending toward the nasal cavity of the patient, the second set of secondary conduits 106 extending toward the oral cavity of the patient, and the plurality of oxygen delivery ports 108, including the nasal oxygen delivery ports 110 and the oral oxygen delivery ports 112. The cannula 200 may also include the exhalation conduit 114 (e.g., second conduit system, exhalation conduit system) configured to receive exhaled breath from the patient's oral and nasal cavities and direct the exhaled breath to the gas analyzer 20 via the sampling line 18. Thus, the cannula 200 may also include the primary conduit 116, the first set of secondary conduits 118 extending through the channels 64 of the nasal prongs 60, the additional secondary conduit 120 extending through the neck 63 of the oral scoop 62, the funnel 124, and the openings 126.

In certain embodiments, the cannula 200 may also include a pair of slides 202 (e.g., plates) extending from the housing 52 toward the oral scoop 62. As illustrated in FIG. 5B, the slides 202 may be oriented at an angle 204 (e.g., non-zero angle) relative to the vertical axis 90 of the cannula 200, such that oxygen directed from the oral oxygen delivery ports 112 in a direction (e.g., downward direction) along the vertical axis 90 may impinge upon the slides 202 and be directed at an angle toward (e.g., across) the scoop 62 and toward the oral cavity of the patient. In this way, oxygen directed from the oral oxygen delivery ports 112 may be directed across a portion of the oral scoop 62 instead of being directed directly into the oral scoop 62. That is, by employing the slides 202, oxygen delivered via the oral oxygen delivery ports 112 may be directed at least partially along the longitudinal axis 94 toward the oral cavity of the patient, and an amount of oxygen entrained into the exhalation conduit 114 may be limited during an exhalation phase of the patient.

In certain embodiments, the slides 202 may be configured to reduce a flow rate or velocity of the oxygen directed out of the oral oxygen delivery ports 112. In this way, as a patient exhales, the flow rate or pressure of the patient's exhaled breath may more easily bias oxygen away from the oral scoop 62 and/or the exhalation conduit 114, thereby limiting an amount of dilution of the exhaled breath entrained into the exhalation conduit 114. That is, by reducing the flow rate of oxygen using the slides 202, redirection of the oxygen via the patient's exhaled breath may be more easily achieved due to the reduced flow rate or velocity of the oxygen from the oral oxygen delivery ports 112.

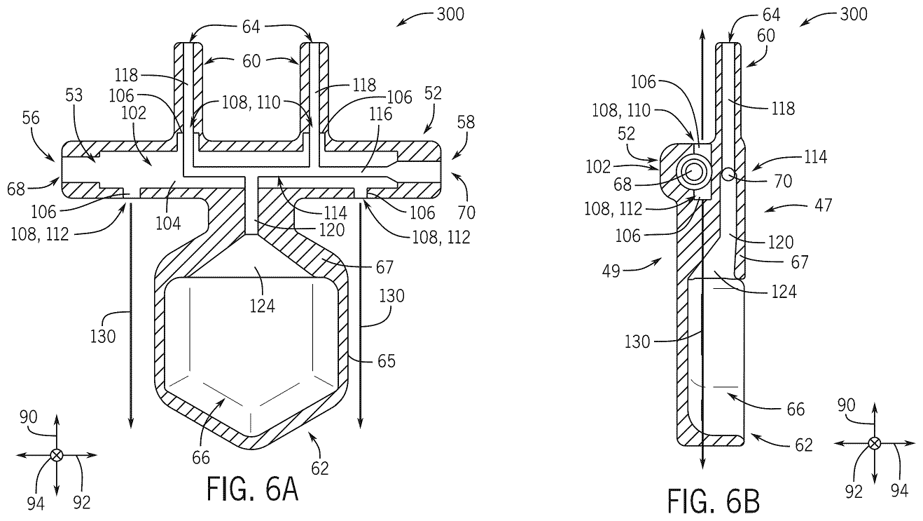

FIGS. 6A and 6B illustrate an embodiment of a cannula 300 (e.g., cannula 14, cannula 45) configured to deliver oxygen to a patient's oral and nasal cavities and having a closed scoop arrangement in which oxygen is routed outside of the scoop 62. The cannula 300 may include similar features to the cannula 45. For example, the cannula 300 may include the plurality of sides 54 that define the housing 52 and the interior space 53, the first end 56, the second end 58, the nasal prongs 60, the oral scoop 62, the channels 64, the body 65, the cavity 66, the lip portion 67, the intake inlet 68 disposed at the first end 56 of the housing 52, and the sampling outlet 70 disposed at the second end 58 of the housing 52.

Additionally, the cannula 300 may include similar features to the cannula 100. For example, the cannula 300 may include the oxygen delivery conduit 102 (e.g., second conduit system, oxygen delivery conduit system) configured to receive oxygen from the oxygen source 16 and deliver the oxygen to the patient's oral and nasal cavities. Thus, the cannula 300 may include the primary conduit 104, the first set of secondary conduits 106 extending toward the nasal cavity of the patient, the second set of secondary conduits 106 extending toward the oral cavity of the patient, and the plurality of oxygen delivery ports 108, including the nasal oxygen delivery ports 110 and the oral oxygen delivery ports 112. The cannula 300 may also include the exhalation conduit 114 (e.g., second conduit system, exhalation conduit system) configured to receive exhaled breath from the patient's oral and nasal cavities and direct the exhaled breath to the gas analyzer 20 via the sampling line 18. Thus, the cannula 300 may also include the primary conduit 116, the first set of secondary conduits 118 extending through the channels 64 of the nasal prongs 60, the additional secondary conduit 120 extending through the neck 63 of the oral scoop 62, and the funnel 124.

In the illustrated example, the oral oxygen delivery ports 112 of the cannula 300 may not align with the oral scoop 62 in a direction along the vertical axis 90 of the cannula 300. Instead, the oral oxygen delivery ports 112 of the cannula 300 may be oriented such that oxygen directed out of the oral oxygen delivery ports 112 is directed around or outside of the oral scoop 62 instead of into the oral scoop 62. For example, as illustrated in FIG. 6A, each of the oral oxygen delivery ports 112 may be offset along the lateral axis 92 of the cannula 300 relative to the oral scoop 62 such that oxygen is delivered around the oral scoop and into the oral cavity of the patient. Additionally, because the oral oxygen delivery ports 112 of the cannula 300 are not configured to direct oxygen into the oral scoop 62, the oral scoop 62 of the cannula 300 may not include the openings 126 (see FIGS. 3A, 5A). By directing the oxygen around the oral scoop 62, the cannula 300 may limit an amount of dilution of the exhaled breath received by the exhalation conduit 114.

FIG. 6B is a side view showing oxygen delivery. In contrast to the arrangement shown in FIG. 5B in which oxygen is routed inside of the scoop 62, the oxygen in FIG. 6B is shown as being delivered downward and around the scoop 62. Returning to FIG. 6A, the oxygen delivery ports 112 are also offset from the nasal delivery ports 110 along the lateral axis 92. The oxygen delivery ports 112 flank the nasal delivery ports 110, such that the nasal delivery ports 110 are positioned between the oxygen delivery ports 112, which are closer to the respective ends 56, 58 of the cannula 300. In the illustrated arrangement, the oxygen delivery ports 112 may be positioned relatively closer to the ends 56, 58 to ensure that the scoop 62 does not obstruct oxygen delivery.

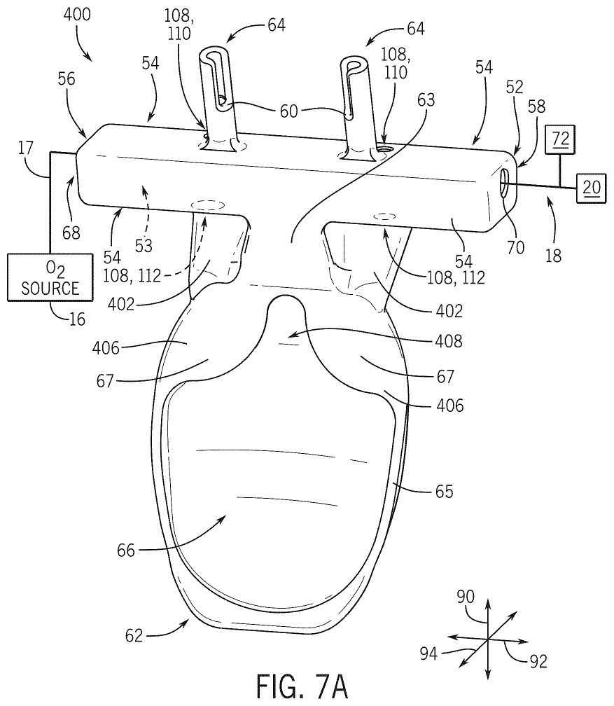

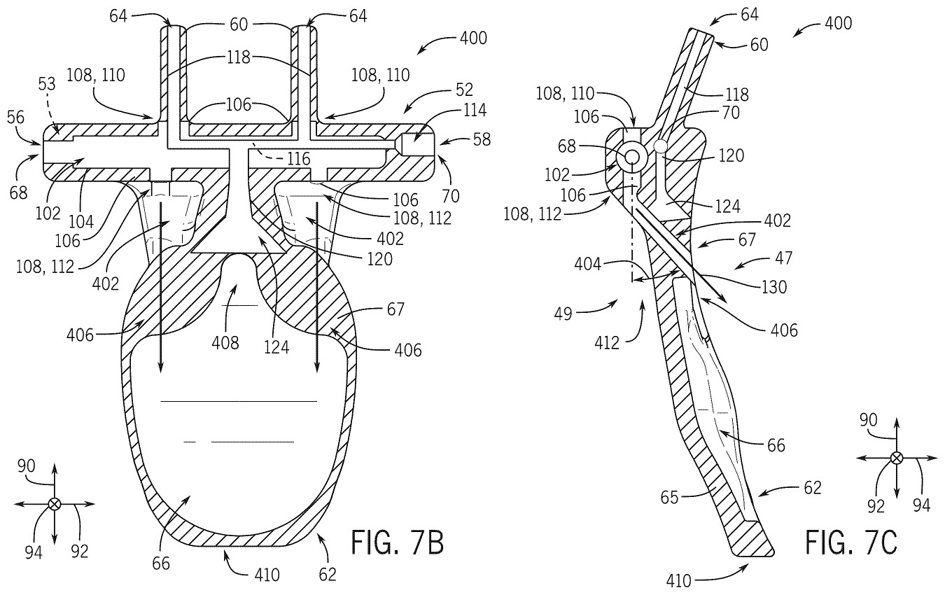

FIGS. 7A, 7B, and 7C illustrate an embodiment of a cannula 400 (e.g., cannula 14, cannula 45) configured to deliver oxygen to a patient's oral and nasal cavities. The cannula 400 may include similar features to the cannula 45. For example, the cannula 400 may include the plurality of sides 54 that define the housing 52 and the interior space 53, the first end 56, the second end 58, the nasal prongs 60, the oral scoop 62, the channels 64, the body 65, the cavity 66, the lip portion 67, the intake inlet 68 disposed at the first end 56 of the housing 52, and the sampling outlet 70 disposed at the second end 58 of the housing 52.

Additionally, the cannula 400 may include similar features to the cannulas 100, 200, and 300. For example, the cannula 400 may include the oxygen delivery conduit 102 (e.g., second conduit system, oxygen delivery conduit system) configured to receive oxygen from the oxygen source 16 and deliver the oxygen to the patient's oral and nasal cavities. Thus, the cannula 400 may include the primary conduit 104, the first set of secondary conduits 106 extending toward the nasal cavity of the patient, the second set of secondary conduits 106 extending toward the oral cavity of the patient, and the plurality of oxygen delivery ports 108, including the nasal oxygen delivery ports 110 and the oral oxygen delivery ports 112. The cannula 400 may also include the exhalation conduit 114 (e.g., second conduit system, exhalation conduit system) configured to receive exhaled breath from the patient's oral and nasal cavities and direct the exhaled breath to the gas analyzer 20 via the sampling line 18. Thus, the cannula 200 may also include the primary conduit 116, the first set of secondary conduits 118 extending through the channels 64 of the nasal prongs 60, the additional secondary conduit 120 extending through the neck 63 of the oral scoop 62, and the funnel 124.

In certain embodiments, similar to the cannula 200, the cannula 400 may include a pair of slides 402 (e.g., plates) extending from the housing 52 toward the oral scoop 62. As illustrated in FIG. 7C, the slides 402 may be oriented at an angle 404 (e.g., non-zero angle) relative to the vertical axis 90, such that oxygen directed from the oral oxygen delivery ports 112 in a direction (e.g., downward direction) along the vertical axis 90 may impinge upon the slides 402 and be directed at an angle toward (e.g., across) the scoop 62 and toward the oral cavity of the patient. In this way, oxygen directed from the oral oxygen delivery ports 112 may be directed across a portion (e.g., the lip portion 67, an exterior surface portion) of the oral scoop 62 instead of being directed directly into the oral scoop 62. That is, by employing the slides 402, oxygen delivered via the oral oxygen delivery ports 112 may be directed at least partially along the longitudinal axis 94 toward the oral cavity of the patient, and an amount of oxygen entrained into the exhalation conduit 114 may be limited during an exhalation phase of the patient.

Additionally, as illustrated in FIGS. 7A, 7B, and 7C, the cannula 400 may not include the openings 126, and instead, the pair of slides 402 may be configured to direct the oxygen delivered via the oral oxygen delivery ports 112 across a surface of the lip portion 67 of the cannula 400 and into the oral cavity of the patient. For example, in certain embodiments, the lip portion 67 of the cannula 400 may include a pair of lobe portions 406 (e.g., lobes, lobe surfaces) and a recess 408 disposed between and separating the lobe portions 406. Each lobe portion 406 may align with a corresponding slide 402 (e.g., relative to the lateral axis 92) and may be positioned proximate a downstream end of the corresponding slide 402 (e.g., relative to the flow direction of oxygen through the oral oxygen delivery ports 112) such that oxygen delivered via the oral oxygen delivery ports 112 may be directed across the lobe portions 406 (e.g., across an exterior surface of the lobe portions 406). In certain embodiments, the lobe portions 406 may further define the cavity 66 of the oral scoop 62, thereby facilitating capture of oral exhaled breath from the patient.

In certain embodiments, the lobe portions 406 may function as an extension of the slides 402. For example, similar to the slides 402, each lobe portion 406 may be oriented at an angle relative to the vertical axis 90 (e.g., in a direction toward the oral cavity of the patient) such that oxygen delivered via the oral oxygen delivery ports 112 may be directed toward the oral cavity of the patient. As such, in certain embodiments, a length of the slides 402 may be less than a length of the slides 202, and the angle 404 at which the slides 402 extend relative to the vertical axis 90 may be greater than the angle 202 at which the slides 202 extend relative to the vertical axis 90. In certain embodiments, the angle 404 at which the slides 402 extend from the vertical axis 90 may be within a threshold degree of similarity (e.g., within 5 degrees, 10 degrees, 20 degrees) relative to the angle at which the lobe portions 406 of the oral scoop 62 extend relative to the vertical axis 90 to facilitate the delivery of oxygen into the oral cavity of the patient. It should be appreciated that the lip portion 67 of the cannula 400 (e.g., a lip portion 67 having the lobe portions 406, 408) may be applied to any of the cannulas 45, 100, 200, 300, and 400 discussed herein.

In certain embodiments, the slides 402 and/or the lobe portions 406 may be configured to reduce a flow rate or velocity of the oxygen directed out of the oral oxygen delivery ports 112. In this way, as a patient exhales, the flow rate or pressure of the patient's exhaled breath may more easily bias oxygen away from the oral scoop 62 and/or the exhalation conduit 114, thereby limiting an amount of dilution of the exhaled breath entrained into the exhalation conduit 114. That is, by reducing the flow rate of oxygen using the slides 402 and/or the lobes 406 of the lip portion 67, redirection of the oxygen via the patient's exhaled breath may be more easily achieved due to the reduced flow rate or velocity of the oxygen from the oral oxygen delivery ports 112.

As noted above, in certain embodiments, the oral scoop 62 may extend from the housing 52 in a direction (e.g., downward direction) at an angle relative to the vertical axis 90 such that the oral scoop 62 extends toward the oral cavity of the patient. For example, as illustrated in FIG. 7C, a bottom portion 410 of the oral scoop 62 may be closer to a patient relative to an upper portion 412 of the oral scoop 62 in an assembled configuration of the cannula 400. In this way, an increased amount of oxygen delivered via the oral oxygen delivery ports 112 may be contained within the cavity 66 and/or directed into the oral cavity of the patient during an inhalation phase of the patient.

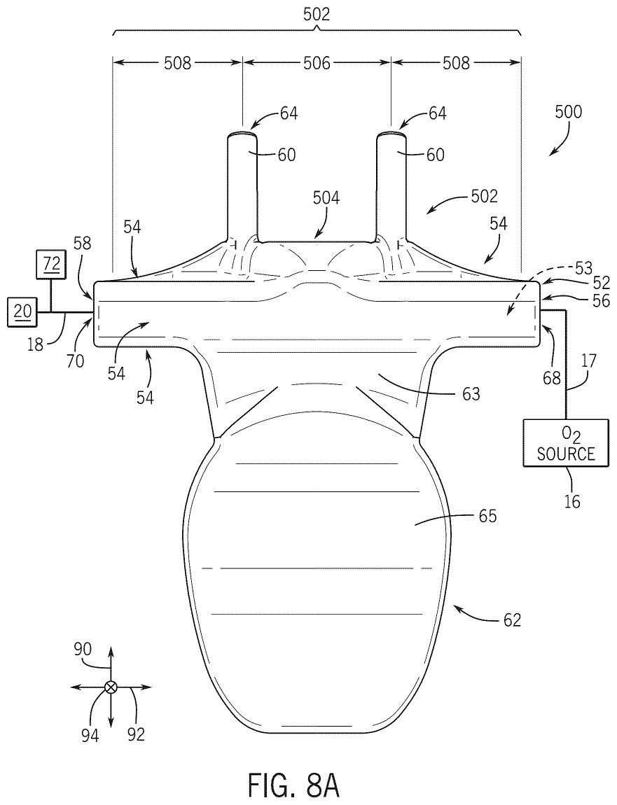

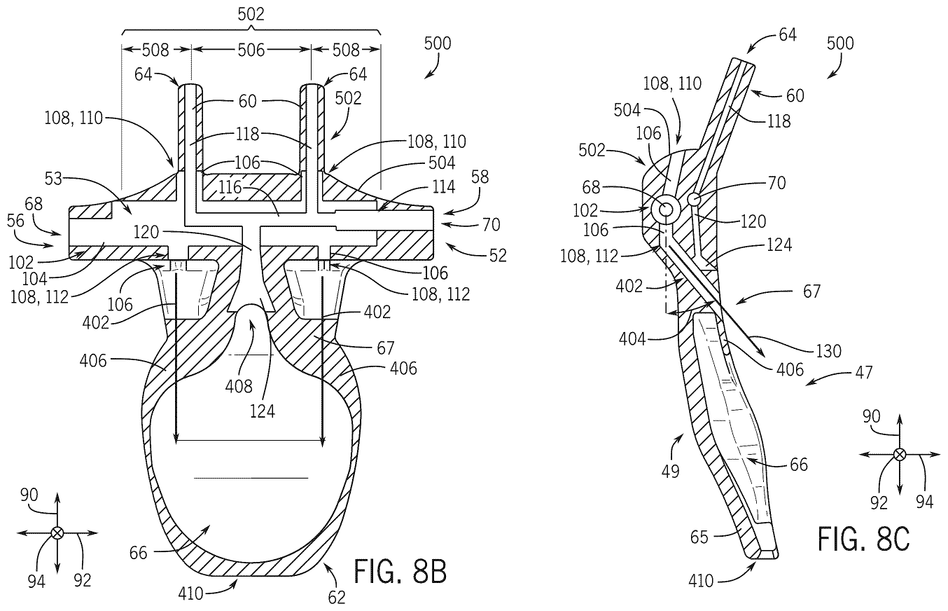

FIGS. 8A, 8B, and 8C illustrate an embodiment of a cannula 500 (e.g., cannula 14, cannula 45) configured to deliver oxygen to a patient's oral and nasal cavities. The cannula 500 may include similar features to the cannulas 45. For example, the cannula 500 may include the plurality of sides 54 that define the housing 52 and the interior space 53, the first end 56, the second end 58, the nasal prongs 60, the oral scoop 62, the channels 64, the body 65, the cavity 66, the lip portion 67, the intake inlet disposed at the first end 56 of the housing 52, and the sampling outlet 70 disposed at the second end of the housing 52.

Additionally, the cannula 500 may include similar features to the cannulas 100, 200, 300, and 400. For example, the cannula 500 may include the oxygen delivery conduit 102 (e.g., second conduit system, oxygen delivery conduit system) configured to receive oxygen from the oxygen source 16 and deliver the oxygen to the patient's oral and nasal cavities. Thus, the cannula 500 may include the primary conduit 104, the first set of secondary conduits 106 extending toward the nasal cavity of the patient, the second set of secondary conduits 106 extending toward the oral cavity of the patient, and the plurality of oxygen delivery ports 108, including the nasal oxygen delivery ports 110 and the oral oxygen delivery ports 112. The cannula 500 may also include the exhalation conduit 114 (e.g., second conduit system, exhalation conduit system) configured to receive exhaled breath from the patient's oral and nasal cavities and direct the exhaled breath to the gas analyzer 20 via the sampling line 18. Thus, the cannula 500 may also include the primary conduit 116, the first set of secondary conduits 118 extending through the channels 64 of the nasal prongs 60, the additional secondary conduit 120 extending through the neck 63 of the oral scoop 62, and the funnel 124.

In certain embodiments, the cannula 500 may also include features similar to the cannula 400. For example, the cannula 500 may include the pair of slides 402 extending from the housing 52 toward the oral scoop 62. As discussed above, the slides 402 may extend from the housing 52 at the angle 404 relative to the vertical axis 90, thereby enabling oxygen directed from the oral oxygen delivery ports 112 to impinge upon the slides 402 and be directed at an angle toward (e.g., across the scoop 62) and toward the oral cavity of the patient. The cannula 500 may also include a lip portion 67 having a pair of lobed portions 406 (e.g., lobes) and a recess 408, and may not include the openings 126. The slides 402 of the cannula 500 may be configured to direct oxygen delivered via the oral oxygen delivery ports 112 across the lip portion 67 and toward the oral cavity of the patient, as discussed above.

As illustrated in FIGS. 8A, 8B, and 8C, the profile of the cannula 500 may include certain features that facilitate oral and nasal oxygen delivery and sampling of exhaled nasal gases and exhaled mouth gases via the cannula 500. For example, a first side 54 (e.g., upper side, top side) of the cannula 500 may include a protrusion 502 extending from the first side 54 in a direction (e.g., vertical direction) along the vertical axis 90 toward the nasal cavity of the patient. The nasal prongs 60 may extend from the protrusion 502 and the first set of secondary conduits 118 may extend through the protrusion 502 and through the channels 64 of the nasal prongs 60, thereby enabling the exhalation conduit 114 to receive nasal exhaled breath from the patient, as described above.

The protrusion 502 may define a contoured profile 504 of the cannula 500 configured to engage with aspects of the nasal cavity of the patient, thereby facilitating oral and nasal oxygen delivery and sampling of exhaled gases (e.g., nasal and mouth) via the cannula 500. For example, the contoured profile 504 may include a primary portion 506 (e.g., bridge portion) configured to support (e.g., engage) a nasal base (e.g., septum) of the nasal cavity of the patient, and a pair of secondary portions 508 (e.g., wing portions, wings). Each secondary portion 508 may extend from the primary portion 506 at an angle (e.g., non-zero) angle toward the housing 52 and may be configured to support other aspects (e.g., nostril) of the nasal cavity of the patient. For example, the secondary portions 508 may engage with the nostrils of the patient and may be configured to limit an amount of rotation of the cannula 500 relative to the patient.

By employing the protrusion 502, a degree of opening of the nasal cavity of the patient utilizing the cannula 500 may be increased relative to traditional cannulas. For example, each secondary portion 508 of the contoured profile 504 may diverge away from the nasal cavity of the patient (e.g., in a downward direction at an angle toward the housing 52) at a point 510 proximate the corresponding nasal prong 60. As the secondary portion 508 extends away from the nasal cavity of the patient, clearance may be provided between the contoured profile 504 and a portion of the patient's nasal cavity that extends beyond the point 510 at which the secondary portion 508 begins to diverge. In this way, an amount of pressure associated with delivery of the oxygen into the nasal cavity of the patient may be reduced, mixing of delivered oxygen and exhaled nasal gases may be reduced, and nasal oxygen delivery and sampling of nasal exhaled gases may be improved. In certain embodiments, features of the cannula 500 may be composed of different materials having different characteristics (e.g., elasticity, stiffness). For example, in certain embodiments, components that interface with (e.g., contact, directly contact) the patient, such as the protrusion 504, may be composed of a material having a lower stiffness relative to the material of the plurality of sides 54. In this way, an amount of support and/or comfort may be increased relative to traditional cannulas. Alternatively, support and/or comfort may be provided by designing the features of the cannula 500 with a single material having a desired thickness and/or shape to provide the desired characteristics (e.g. stiffness, elasticity). For example, in certain embodiments, components that interface with (e.g., contact, directly contact) the patient, such as the protrusion 504, may have lower thickness and/or bowed shape, thereby resulting in a lower stiffness relative to the material of the plurality of sides 54.

It should be appreciated that while the cannulas 200, 300, 400, and 500 may include certain features that differ from the cannula 100 described above, the movement of delivered gas and exhaled gas through the oral scoop 62 of the cannulas 200, 300, 400, and 500 during inhalation and exhalation may be similar to that described above with respect to FIGS. 4A and 4B (e.g., may be similar to the movement of delivered gas and exhaled gas through the oral scoop 62 of the cannula 100). For example, while the cannulas 200, 400, and 500 may include slides 202, and 402, respectively, and while cannulas 300, 400, and 500 may not include the openings 126, the flow of oxygen may be delivered (e.g., at a desired flow rate rate) toward the oral scoop 62 (e.g., via a continuous oxygen delivery). In embodiments of a cannula including a pair of slides (e.g., cannulas 200, 400, 500), the slides 202, 402 may be oriented at the angle 204, 404, respectively, such that oxygen delivered out of the oral oxygen delivery ports 112 may impinge upon the slides 202, 402 and be directed at an angle toward the cavity 66 and toward the oral cavity of the patient.

The flow of oxygen from the oral oxygen delivery ports 112 in the cannulas 200, 300, 400, and 500 may be overcome by a pressure of the exhaled breath from the patient, and thus, the desired flow rate of oxygen through the oral oxygen delivery ports 112 may be based on an expected pressure of flow of an exhaled breath from an oral cavity of the patient, as described above with respect to FIG. 4B. That is, the pressure of exhaled breath from the patient's oral cavity may be greater than the pressure of oxygen delivered to the oral cavity of the patient via the oral oxygen delivery ports 112. In this way, any oxygen which may be present within the oral scoop 62 of the cannulas 200, 300, 400 and 500 during an exhalation phase of a patient may be redirected or biased out of the oral scoop 62, thereby limiting dilution of the exhaled breath received by the exhalation conduit 114 and delivered to the gas analyzer 20. Further, the sampled exhaled breath does not mix with incoming delivered oxygen during exhalation. In this manner, oxygen influx into the scoop 62 is temporarily overcome by the exhaled breath pushing the oxygen out and around the scoop 62. This serves to separate the sampled exhaled breath from the incoming flow of oxygen, permitting sampling of exhaled breath from the mouth while simultaneously delivering oxygen towards the mouth in the cannulas 200, 300, 400, and 500.

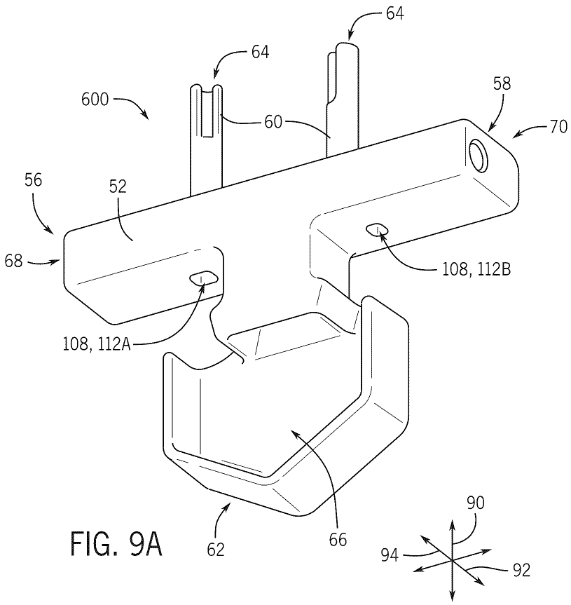

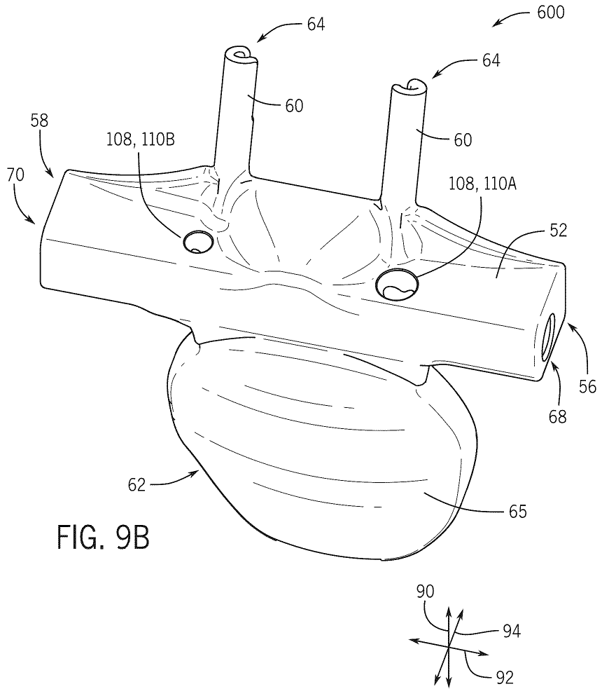

FIG. 9A illustrates a bottom perspective view of the oral oxygen delivery ports 112 and FIG. 9B illustrates a top perspective view of the nasal oxygen delivery ports 110 of a cannula 600. The cannula 600 may be representative of any of the various cannulas described herein (e.g., cannula 14, cannula 45, cannula 100, cannula 200, cannula 300, cannula 400, cannula 500). In certain embodiments, each of the nasal oxygen delivery ports 110 may be sized differently (e.g., relative to one another) and each of the oral oxygen delivery ports 112 may be sized differently to facilitate distribution of oxygen to the patient's oral and nasal cavities. That is, each of the oral oxygen delivery ports 112 may be sized asymmetrically such that the flow of oxygen from each of the oral oxygen delivery ports 112 is substantially symmetrical (e.g., substantially the same). Similarly, in certain embodiments, each of the nasal oxygen delivery ports 110 may be sized asymmetrically such that the flow of oxygen from each of the nasal oxygen delivery ports 110 is substantially symmetrical (e.g., substantially the same).

For example, as shown in FIG. 9A, a first oral oxygen delivery port 112A positioned proximate the first end 56 of the cannula 600 may be larger than a second oral oxygen delivery port 112B positioned proximate the second end 58 of the cannula 600. Similarly, as shown in FIG. 9B, a first nasal oxygen delivery port 110A positioned proximate the first end 56 of the cannula 600 may be larger than a second nasal oxygen delivery port 110B positioned proximate the second end 58 of the cannula 600. By sizing the oral oxygen delivery ports 112A, 112B differently, and the nasal oxygen delivery ports 110A, 110B differently, the flow rate and velocity of the oxygen directed through the oral oxygen delivery ports 112 and the nasal oxygen delivery ports 110 may be controlled to permit generally symmetrical distribution of oxygen flow via asymmetrically-sized and spaced apart nasal and oral oxygen delivery ports 110, 112.

In an embodiment, the difference in size between the ports 112A, 112B and/or a difference in size between the ports 110A, 110B may be a total area of first oral oxygen delivery port 112A or the first nasal oxygen delivery port 110A relative to the second oral oxygen delivery port 112B or the second nasal oxygen delivery port 110B, respectively. The area of the first oral oxygen delivery port 112A or the first nasal oxygen delivery port 110A may be at least 1.1, 1.3, 1.5, 2, or 5 times larger than an area of the second oral oxygen delivery port 112B or the second nasal oxygen delivery port 110B, respectively. In an embodiment, a diameter of first oral oxygen delivery port 112A or the first nasal oxygen delivery port 110A may be at least 1.1, 1.3, 1.5, 2, or 5 times larger than a diameter of the second oral oxygen delivery port 112B or the second nasal oxygen delivery port 110B, respectively. In an embodiment, the first oral oxygen delivery port 112A or the first nasal oxygen delivery port 110A may be shaped differently than the second oral oxygen delivery port 112B or the second nasal oxygen delivery port 110B, respectively. For example, the second oxygen delivery port 112B may be a circle, while the first oral oxygen delivery port 112A may have an elongated ovoid shape. It should be appreciated that each of the above described sizes, areas, diameters, shapes may be selected to provide a desired flow rate (e.g., desired volume) of oxygen to a patient.

As noted above, by employing oxygen delivery ports having different sizes, symmetrical distribution of oxygen between a respective type of oxygen delivery port (e.g., symmetrical distribution between nasal oxygen delivery ports 110A and 110B, symmetrical distribution of oxygen between oral oxygen delivery ports 112A and 112) may be achieved. For example, in an embodiment in which the ports 112A, 112B are similarly sized, the flow rate of oxygen within the oxygen delivery conduit 102 may cause the flow of oxygen to bypass the first oral oxygen delivery port 112A and travel toward a distal end (e.g., end of the oxygen delivery conduit proximate the second end 58 of the housing 52) of the oxygen delivery conduit 102. As the oxygen travels toward the distal end of the oxygen delivery conduit 102, a first portion of the oxygen may be discharged out of the oxygen delivery conduit 102 via the second oral oxygen delivery port 112B. A second portion of the oxygen may impinge against the inner walls of the oxygen delivery conduit 102 before being redirected in an upstream direction (e.g., relative to the flow of oxygen into the oxygen delivery conduit 102), thereby creating turbulence and/or an increase in pressure within the oral oxygen delivery conduit 102. The turbulence and/or increase in pressure may enable some oxygen to be discharged from the oral oxygen delivery conduit 102 via the first oral oxygen delivery port 112A. However, the flow rates between similarly sized ports 112A, 112B may diverge as a result of the flow dynamics within the oxygen delivery conduit 102. It should be appreciated that the same fluid flow dynamics discussed above may apply to similarly sized nasal oxygen delivery ports 110.

Conversely, in embodiments in which the oral oxygen delivery ports 112A, 112B are sized differently and/or the nasal oxygen delivery ports 110A, 110B are sized differently, oxygen may flow substantially symmetrically between the oral oxygen delivery ports 112A, 112B and the nasal oxygen delivery ports 110A, 110B, respectively. For example, by increasing the size of the first oral oxygen delivery port 112A relative to the second oral oxygen delivery port 112B, an increased amount of oxygen may be discharged from the first oral oxygen delivery port 112A relative to an embodiment in which the oral oxygen delivery ports 112 are similarly sized. That is, by increasing the size of the first oral oxygen delivery port 112A relative to the second oral oxygen delivery port 112B, a decreased amount of oxygen may bypass the first oral oxygen delivery port 112A (e.g., relative to an embodiment in which the oral oxygen delivery ports 112 are similarly sized), and instead, may be delivered to the patient via the first oral oxygen delivery port 112A. Additionally, as a decreased amount of oxygen bypasses the first oral oxygen delivery port 112A, a pressure within the oral oxygen delivery conduit 102 may decrease, thereby decreasing an amount of turbulence within the oral oxygen delivery conduit 102. In this way, symmetrical (e.g., substantially symmetrical) distribution of oxygen between the oral oxygen delivery ports 112 may be more readily achievable. It should be appreciated that the same fluid flow dynamics discussed above may apply to the nasal oxygen delivery ports 110A, 110B.

It should be appreciated that certain aspects of the cannulas discussed herein may be modified and/or adjusted to accommodate patients having an array of physical characteristics. For example, a respective positioning and/or sizing of the various components of the cannulas discussed herein may be modified based on an age of the patient and/or other physical characteristics of the patient (e.g., facial geometry) without departing from the scope of this disclosure.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

1. A cannula for exhaled gas monitoring and oxygen delivery, comprising:
an oxygen delivery conduit entering the cannula;
an exhalation conduit exiting the cannula;
first and second nasal prongs each comprising a channel directing exhaled nasal breath into the exhalation conduit;
an oral scoop forming a cavity that is fluidly coupled to the exhalation conduit; and
first and second ports fluidly coupled to the oxygen delivery conduit and opening toward the oral scoop.

2. The cannula of claim 1, comprising a plurality of sides defining a housing of the cannula, wherein the oxygen delivery conduit and the exhalation conduit are disposed within an interior space of the housing, wherein a first side of the plurality of sides comprises a protrusion extending from the housing and configured to support a nasal cavity in an assembled configuration of the cannula.

3. The cannula of claim 2, wherein the protrusion comprises:
a first portion extending between the first and second nasal prongs; and
a first wing portion extending from the first portion at an angle toward the housing; and
a second wing portion extending from the first portion at an additional angle toward the housing.

4. The cannula of claim 2, wherein the protrusion is configured to increase a degree of opening of the nasal cavity.

5. The cannula of claim 1, wherein the oxygen delivery conduit and the exhalation conduit are fluidically isolated from one another.

6. The cannula of claim 1, wherein the first and second ports substantially align with the oral scoop along a vertical axis of the cannula such that oxygen is delivered into the oral scoop via openings along a side of the oral scoop.

7. The cannula of claim 1, wherein the cannula comprises first and second plates, wherein the first and second ports direct oxygen toward the first and second plates, and wherein the first and second plates direct the oxygen at an angle across the oral scoop.

8. The cannula of claim 1, wherein the first and second ports are offset from the oral scoop along a vertical axis of the cannula such that oxygen is delivered outside of the oral scoop.

9. The cannula of claim 1, wherein the first and second ports are first and second oral oxygen delivery ports, and wherein the cannula comprises first and second nasal oxygen delivery ports fluidly coupled to the oxygen delivery conduit and opening away from the oral scoop.

10. The cannula of claim 9, wherein the first and second nasal oxygen delivery ports flank the first and second oral oxygen delivery ports along a lateral axis of the cannula.

11. A capnography system, comprising:
a cannula comprising:
an oxygen delivery conduit entering the cannula;
an exhalation conduit exiting the cannula;
an oral scoop forming a cavity that is fluidly coupled to the exhalation conduit; and
first and second oral oxygen delivery ports fluidly coupled to the oxygen delivery conduit and opening toward the oral scoop; and
a monitor fluidly coupled to the exhalation conduit to analyze exhaled carbon dioxide received via the exhalation conduit.

12. The capnography system of claim 11, wherein the cannula comprises:
first and second nasal prongs each comprising a channel directing exhaled nasal breath into the exhalation conduit.

13. The capnography system of claim 11, wherein the first and second oral oxygen delivery ports deliver oxygen into the oral scoop.

14. The capnography system of claim 11, wherein the cannula comprises first and second angled plates each receiving oxygen from the first and second oral oxygen delivery ports and directing the oxygen at an angle across the oral scoop.

15. The capnography system of claim 11, wherein the first and second oral oxygen delivery ports deliver oxygen outside of the oral scoop.

16. The capnography system of claim 11, wherein a portion of the exhalation conduit extends from the cannula and terminates at or in the oral scoop to receive oral exhaled breath.

17. The capnography system of claim 11, wherein the first oral oxygen delivery port is a different size than the second oral oxygen delivery port.

18. The capnography system of claim 11, wherein the cannula comprises:
first and second nasal oxygen delivery ports fluidly coupled to the oxygen delivery conduit, wherein the first nasal oxygen delivery port is a different size than the second nasal oxygen delivery port.

19. A method of capnography, comprising:
receiving, via an oral scoop of a cannula, an exhaled breath from a patient, wherein the exhaled breath comprises carbon dioxide, and wherein the oral scoop is positioned proximate an oral cavity of the patient in an installed configuration of the cannula device;
directing, via a sampling line coupled to the cannula device, the exhaled breath to a gas analyzer; and
delivering, via first and second oral oxygen delivery ports of the cannula device, oxygen from an oxygen source to the oral cavity of the patient while receiving the exhaled breath.

20. The method of claim 18, wherein delivering the oxygen comprises delivering the oxygen at a rate of at least 10 L/minute.
